(19) United States
(12) Patent Application Publication
(10) Pub. No.: US 2011/0054592 A1
(43) Pub. Date: Mar. 3, 2011

(54) FLEXIBLE EXPANDABLE STENT AND METHODS OF DEPLOYMENT

(75) Inventor: Thilo U. Fliedner, Niederpocking (DE)

(73) Assignee: CORNOVA, INC., Burlington, MA (US)

(21) Appl. No.: 12/747,376

(22) PCT Filed: Dec. 10, 2008
(86) PCT No.: PCT/US08/86267

§ 371 Date: Sep. 16, 2010

Related U.S. Application Data
(60) Provisional application No. 61/013,246, filed on Dec. 12, 2007.

(51) Int. Cl. A61F 2/82

(52) U.S. Cl. 623/1.16

(57) ABSTRACT

A flexible, expandable stent assembly comprises a pattern of interconnected struts along a curvilinear path. The struts define a cylindrically shaped channel that extends along a longitudinal axis. The channel has a plurality of openings. The struts comprise a plurality of circumferential arrays of webs or bends. Each circumferential array is connected to an adjacent circumferential array by fewer than four cross-links. Each cross-link extending from a first side of a circumferential array of the plurality of circumferential arrays is substantially circumferentially offset from every cross-link extending from an opposite side of the same circumferential array. Each of the struts has, in a cross-section generally normal to the curvilinear path of the strut and normal to a center of curvature of the channel, a strut surface width that is at least one and a half times that of a strut surface height of the strut.

### RELATED APPLICATIONS

This application is a continuation-in-part of U.S. patent application Ser. No. 11/613,443 filed Dec. 20, 2006, which is a continuation-in-part of U.S. patent application Ser. No. 29/252,668 filed Jan. 25, 2006, issued as U.S. Pat. No. D553,746 and U.S. application Ser. No. 29/252,669 filed Jan. 25, 2006, issued as U.S. Pat. No. D553,747, the contents of each of which are herein incorporated by reference in their entirety. This application claims the benefit of U.S. patent application Ser. No. 61/013,246 filed on Dec. 12, 2007, the contents of which are incorporated herein by reference in its entirety. This application is related to U.S. patent application Ser. No. 11/843,376 filed on Aug. 22, 2007, U.S. patent application Ser. No. 11/843,402 filed on Aug. 22, 2007, U.S. patent application Ser. No. 60/823,692 filed on Aug. 28, 2006, U.S. patent application Ser. No. 60/825,434 filed on Sep. 13, 2006, U.S. patent application Ser. No. 60/895,924 filed on Mar. 20, 2007, U.S. patent application Ser. No. 60/941,813 filed on Jun. 4, 2007, U.S. patent application Ser. No. 60/975,383, filed Sep. 26, 2007, and World International Property Organization (WIPO) International Patent Application Number PCT/US08/77871 filed on Sep. 26, 2008, the contents of each of which are incorporated herein by reference in their entirety.

### BACKGROUND OF THE INVENTION

1. Field of the Invention

Embodiments of the present invention relate to medical stents which are implantable devices for propping open and maintaining the patency of vessels and ducts in the vasculature of a human being.

2. Description of the Related Art

Stents are implantable prosthesis used to maintain and/or reinforce vascular and endoluminal ducts in order to treat and/or prevent a variety of medical conditions. Typical uses include maintaining and supporting coronary arteries after they are opened and unclogged by a medical procedure, such as through an angioplasty operation. A stent is typically deployed in an unexpanded or crimped state using a catheter and, after being properly positioned within a vessel, is then expanded into place.

As a foreign object inserted into a vessel, a stent can potentially impede the flow of blood through the vessel. This effect can also be exacerbated by the undesired growth of tissue on and around the stent, potentially leading to complications including thrombosis and restenosis. Thus, stents are manufactured to minimize impedance of blood flow through a vessel while being capable of effectively maintaining the expanded state of the vessel. Typical stents have the basic form of an open-ended tubular element supported by a mesh of thin struts with openings formed thereinbetween. Such stent designs require excessive amounts of material and excessive strut-to-tissue contact that can increase the likelihood of the above-described complications. These problems can be particular apparent with multiple-stent applications in which the stents overlap each other (e.g., bifurcation procedures).

Thus, many stent designs have been produced to minimize the amount of material used and reduce the level of stent-to-vessel contact percentage, i.e., the percentage of direct strut surface contact relative to the surface area defined by the inner vessel wall along the extent of the stent. Reducing the level of contact reduces the likelihood and level of damage during deployment and adverse reactions caused by implanted materials. Stent designs with insufficient amounts of material, however, and/or with poorly distributed support and expansion profiles can result in complications such as a partial or complete collapse of the struts, and consequently, collapse of portions of the vessel which they support.

For example, some designs included in a category known as “open stent” designs (e.g., having areas of struts with relatively few connecting points) can provide substantial flexibility but may have inadequate support in certain areas of the stent, particularly when placed across hard lesions such as calcified vessels. After an angioplasty balloon is deflated, the stented vessel area will have a tendency to return to its naturally curved state and exert forces on the stent correspondingly. With typical “open” stent designs, the flexing of the stent in response to these forces will generally occur around or pivot about these “open” areas along the limited connecting points, thus potentially opening these “open” areas even further and substantially reducing vessel support about them.

These types of “open” designs are also typified by high proportions of strut deformation, separation, and movement in relatively focused areas of the stent, thus potentially causing high levels of abrasion to adjacent tissue and creating large open unsupported areas across the expanded stent.

Although “open” stent designs can also provide the advantage of reduced strut-to-vessel contact percentage, improvements are needed toward lowering the typical percentage of around 15 percent or more by better distribution of contact along the vessel.

Stents with poorly distributed expansion profiles (e.g., that result in uneven expansion or movement of struts) can potentially cause excessive damage and complicate healing after deployment, increasing the likelihood of restenosis and risky revascularization procedures. For example, a design which is the subject of U.S. Pat. No. 6,432,133 issued Aug. 13, 2002, entitled “Expandable Stents and Method for Making Same,” incorporated herein by reference in its entirety, proposes generally independently expandable radial components with substantially straight longitudinal segments that, during expansion, pivot almost exclusively about a limited set of connecting bends. Thus, the deformation of these stents during expansion would occur almost exclusively by the pivoting and rotation of these straight segments, causing significant movement and abrasion about the adjacent vasculature during expansion.

In addition to strut patterns, the surface profile of a stent strut during and after deployment can impact the level of incidental damage caused to a vessel and effect complications during recovery, including inflammation, restenosis, thrombosis, and the speed of healing about the stent. Particularly sharp or angular strut surfaces that may be adopted to minimize overall stent material can, however, increase the likelihood of adverse complications by causing too much stress and abrasion to the tissue in which the strut surfaces contact. Thus, better optimized strut patterns and strut surface profiles are needed for providing both effective overall support, limiting damage to the tissue during and after deployment, and promoting effective healing.

### SUMMARY OF THE INVENTION

Embodiments of the present invention relate to medical stent assemblies comprised of elongated tubular patterns of metal capable of expanding and propping open a vessel or duct within a living, human being.

In an aspect of the invention, a flexible, expandable, elongated stent assembly is provided having a pattern of interconnected along a curvilinear path, the struts defining a generally cylindrically shaped channel, the channel having a plurality of openings and a longitudinal axis. The struts include a plurality of circumferential arrays of webs or bends of material, each circumferential array connected to an adjacent circumferential array by fewer than 4 cross-links, wherein each circumferential array extending from a first side of a circumferential array of the plurality of circumferential arrays is substantially circumferentially offset from every cross-link extending from an opposite side of the same circumferential array. In a cross-section generally normal to the curvilinear path of the strut and normal to a center of curvature of the channel, the struts have a surface width that is at least one and a half times that of a surface height of the strut.

In an embodiment, the flexible, each circumferential array is connected to an adjacent array by two cross-links.

In an embodiment, the cross-links are arranged such that, upon expansion of said stent assembly from a first position in an unexpanded state to a second position in an unexpanded state, the cross-links are re-oriented or pivoted with respect to the longitudinal axis.

In an embodiment, each of the cross-links is attached to a bend of a circumferential array such that any bending or pivoting of the each of the cross-links is directly and substantially coupled with a bending or pivoting of the attached circumferential array.

In an embodiment, each of the cross-links extends from a mid-portion of a longitudinally extending curved section of a first bend of a first array to a tip portion of a first bend of a longitudinally adjacent second array. In an embodiment, each cross-link connects diagonally positioned bends of said circumferential arrays of bends or webs to each other.

In an embodiment, wherein each cross-link extending from the first side of each circumferential array is substantially is substantially circumferentially offset by at least about 60 degrees from said every cross-link extending from an opposite side of the same circumferential array. In an embodiment, each of the cross-links extending from the first side of each circumferential array is offset by about 90 degrees from said every cross-link extending from an opposite side of the same circumferential array.

In an embodiment, a circumferential gap or open cell is arranged between circumferentially adjacent cross-links, the circumferential gap extending along about a half-circumference of the stent.

In an embodiment, the surface width is greater than about 90 microns. In an embodiment, the surface width is between about 90 and 130 microns. In an embodiment, the surface width is about 120 microns.

In an embodiment, the strut surface width is of about twice that of the strut surface height.

In an embodiment, the stent assembly is manufactured such that upon having an expanded diameter of between about 2.75 mm and 4 mm in a vessel, the stent assembly has less than about 10.5% to 13.5% of strut-to-vessel contact over an area encompassing an entire periphery of the channel.

In an embodiment, the circumferential arrays include arcuately shaped, generally hairpin-like smoothly curved webs or bends. In an embodiment, a substantial portion of each of the arcuately shaped, generally hairpin-like curved webs or bends form arcs of generally the same orientation with respect to the circumference of said stent assembly.

In an embodiment, each of the circumferential arrays of webs or bends comprises a first pattern of lengthwise-sized bends and a second pattern of lengthwise-elongatedly-sized bends at regular intervals on each circumferential array.

In an embodiment, wherein a first stent assembly of the flexible, expandable stent assembly is combined with a second stent assembly that extends at least partway through an opening of the first stent assembly. In an embodiment, the second stent assembly extends at least partway through a generally circumferentially disposed opening of the first stent assembly. In an embodiment, the second stent assembly extends through a generally longitudinally disposed opening of the first stent assembly. In an embodiment, the second stent assembly is of at least one of a smaller length and a smaller diameter than that of the first stent assembly.

In an embodiment, the plurality of circumferential arrays of webs or bends of material is metal.

In an aspect of the invention, a method for expanding and supporting the vasculature of a patient is provided, the method including the step of placing a first stent assembly into a vessel of the patient. The stent assembly includes a pattern of interconnected struts along a curvilinear path, the struts defining a generally cylindrically shaped channel that extends along a longitudinal axis, the channel having a plurality of openings, the struts including a plurality of circumferential arrays of webs or bends of a material, wherein each cross-link extending from one side of a circumferential array of the plurality of circumferential arrays is substantially circumferentially offset from every cross-link extending from an opposite side of the same circumferential array, each circumferential array connected to an adjacent circumferential array by fewer than four cross-links. Each strut has, in a cross-section generally normal to the curvilinear path of the strut and normal to to a center of curvature of the channel, a strut surface width of at least one and a half times that of a strut surface height of the strut.

In an embodiment, the fewer than four cross-links consists of two cross-links.

In an embodiment, a circumferential gap or open cell is arranged between circumferentially adjacent cross-links, the circumferential gap extending along about a half-circumference of the stent assembly.

In an embodiment, the method further includes the step of expanding the first stent assembly.

In an embodiment, the step of expanding the first stent includes re-orienting or pivoting the cross-links with respect to said longitudinal axis.

In an embodiment, each one of the cross-links is fixed to a bend of a circumferentially array such that the re-orienting or pivoting of the each one of the cross-links is directly and substantially coupled with a bending or pivoting of said bend of a circumferential array.

In an embodiment, the direct and substantial coupling is provided by a cross-link directly connected between a mid-portion of a longitudinally extending curved section of a bend of a first circumferential array and the tip portion of a bend of a second circumferential array that is adjacent to the first circumferential array.

In an embodiment, the stent is expanded to a diameter between about 2.75 millimeters and 4 millimeters and provides an overall strut-to-vessel contact percentage of less than about 10.5% to 13.5% of vessel area encompassing the periphery of the channel.

In an embodiment, the stent conforms to curves in the vessel of the patient by generally concentrating abending of the stent about the longitudinal axis over portions of the stent where the circumferential position of the cross-links substantially corresponds to apexes of the curves in the vessel.

In an embodiment, wherein each cross-link extending from the first side of each circumferential array is substantially circumferentially offset from every cross-link extending from the opposite side of the circumferential array.

In an embodiment, each cross-link extending from the one side of each circumferential array is offset by about 90 degrees from every cross-link extending from the opposite side of the circumferential array.

In an embodiment, the strut surface width of the stent assembly is greater than about 90 microns. In an embodiment, the strut surface width is between about 90 and 130 microns. In an embodiment, the strut surface width is about 120 microns. In an embodiment, the strut surface width is of about twice that of the strut surface height.

In an embodiment, the stent assembly includes circumferential arrays of switchback webs or bends, wherein the circumferential arrays are connected to one another by an arrangement of cross-links, wherein each of the cross-links comprises a path of curvature that continuously extends a path of curvature of at least one of said bends.

In an embodiment, a substantial portion of each of the webs or bends of the stent assembly form arcs of generally the same orientation with respect to the circumference of the stent assembly.

In an embodiment, each of the circumferential arrays of webs or bends of the stent assembly includes a first pattern of lengthwise sized bends and a second pattern of lengthwise-elongatedly sized bends at regular intervals on each circumferential array.

In an embodiment, the first stent assembly is placed across a first arm of a bifurcated vessel and the method further includes a step of placing a second stent assembly at least partway through a side wall opening of the first stent assembly and into a second arm of the vessel bifurcation.

In an embodiment, the second stent assembly is defined by a pattern of struts essentially equivalent to that of the first stent assembly.

In an embodiment, the method further includes the step of extending the first stent assembly by placing a second stent assembly at least partway through a longitudinal end opening of the first stent assembly.

In an embodiment, the second stent assembly is defined by a pattern of struts essentially equivalent to that of the first stent assembly. In an embodiment, the second stent assembly is of a smaller diameter than that of the first stent assembly.

In an embodiment, the second stent assembly is of a shorter length than that of the first stent assembly.

### BRIEF DESCRIPTION OF THE DRAWINGS

The objects and advantages of the present invention will become more apparent when viewed in conjunction with the following drawings. The drawings are not necessarily to scale, emphasis instead being placed upon illustrating the principles of the invention.

FIG. 1 is an illustrative longitudinal presentation, in a flat or “planar” array, of an unexpanded stent assembly in accordance with embodiments of the invention.

FIG. 2 is a side elevational view of a stent assembly in an embodiment of the present invention in a cylindrical configuration in accordance with embodiments of the invention.

FIG. 3A is an enlarged illustrative view, in plan, of a portion of a circumferential array of arcuately shaped hairpin-like bends of the stent assembly shown in FIG. 1 in accordance with embodiments of the invention.

FIG. 3B is an illustrative side perspective view of a strut section of the assembly shown in FIG. 3A.

FIG. 3C is an illustrative cross-sectional view across lines I-I′ of an embodiment of the stent strut section shown in FIGS. 3A and 3B.

FIG. 4A is an illustrative cross-sectional view of a typical square-shaped stent strut abutting a vessel wall.

FIG. 4B is an illustrative cross-sectional view of a stent strut in accordance with an embodiment of the invention abutting a vessel wall in accordance with embodiments of the invention.

FIG. 5 is a perspective illustrative view of two expanded stent assemblies in accordance with an embodiment of the present invention shown interdigitated in a vessel bifurcation in accordance with embodiments of the invention.

FIG. 6 is a view of an enlarged flattened illustrative pattern of the stent of FIGS. 1 and 2 shown after balloon expansion in accordance with embodiments of the invention.

### DETAILED DESCRIPTION OF EMBODIMENTS OF THE INVENTION

The accompanying drawings are described below, in which example embodiments in accordance with the present invention are shown. Specific structural and functional details disclosed herein are merely representative. This invention may be embodied in many alternate forms and should not be construed as limited to example embodiments set forth herein.

Accordingly, specific embodiments are shown by way of example in the drawings. It should be understood, however, that there is no intent to limit the invention to the particular forms disclosed, but on the contrary, the invention is to cover all modifications, equivalents, and alternatives falling within the spirit and scope of the claims. Like numbers refer to like elements throughout the description of the figures.

It will be understood that, although the terms first, second, etc. may be used herein to describe various elements, these elements should not be limited by these terms. These terms are used to distinguish one element from another. For example, a first element could be termed a second element, and, similarly, a second element could be termed a first element, without departing from the scope of the present disclosure. As used herein, the term “and/or” includes any and all combinations of one or more of the associated listed items.

It will be understood that “adjacent” does not necessarily imply contact but may connote an absence of the same type of element(s) therein between “adjacent” elements.

It will be understood that when an element is referred to as being “on,” “connected to,” or “coupled to” another element, it can be directly on, connected to or coupled to the other element or intervening elements may be present. In contrast, when an element is referred to as being “directly on,” “directly connected to” or “directly coupled to” another element, there are no intervening elements present. Also, when an element is referred to as being “attached to” or “affixed to” another element, there are no intervening elements present. Other words used to describe the relationship between elements should be interpreted in a like fashion (e.g., “between” versus “directly between,” “adjacent” versus “directly adjacent,” etc.).

The terminology used herein is for the purpose of describing particular embodiments and is not intended to be limiting of the invention. As used herein, the singular forms “a,” “an” and “the” are intended to include the plural forms as well, unless the context clearly indicates otherwise. It will be further understood that the terms “comprise,” “comprises,” “comprising,” “include,” “includes” and/or “including,” when used herein, specify the presence of stated features, integers, steps, operations, elements, and/or components, but do not preclude the presence or addition of one or more other features, integers, steps, operations, elements, components, and/or groups thereof.

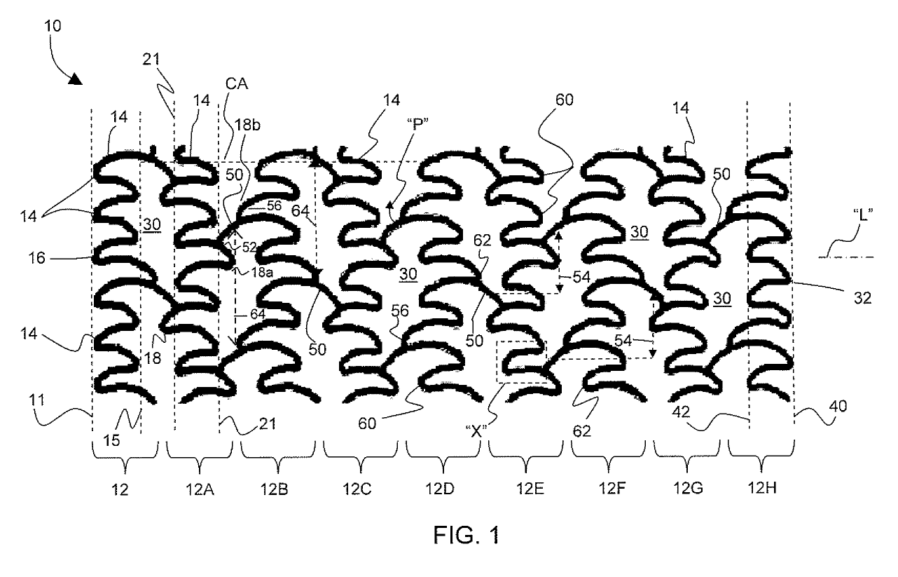

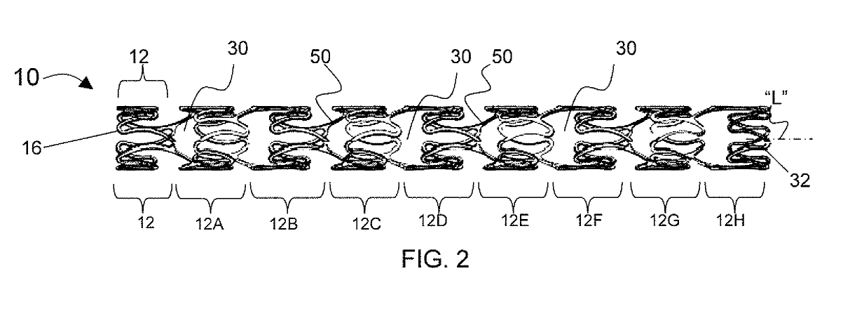

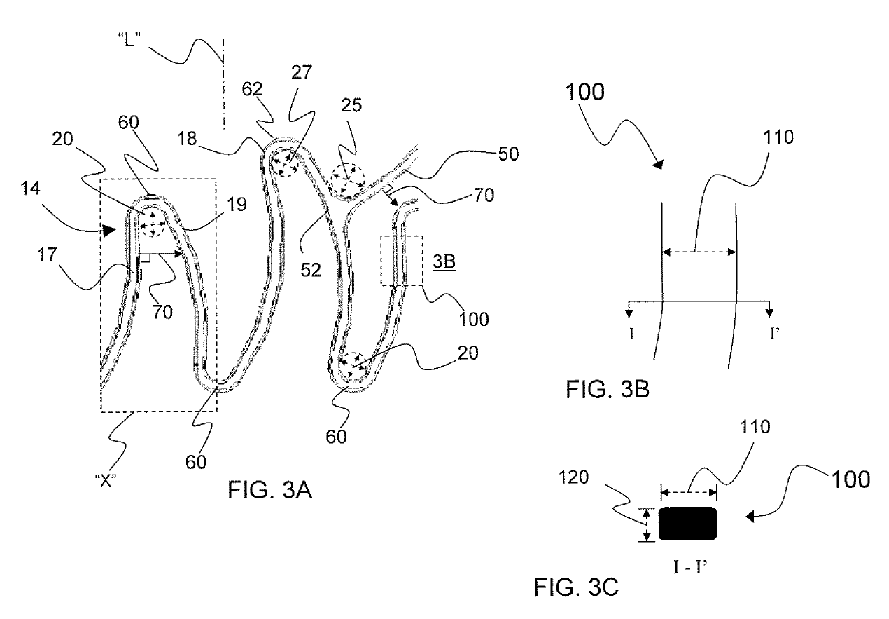

Referring now to the drawings in detail, and particularly to FIG. 1, a medical stent assembly 10 in accordance with an embodiment of the present invention is represented in a flat or planar configuration for ease of understanding. The medical stent assembly 10 is comprised of an elongated tubular pattern of metal capable of expanding and propping open a vessel or duct within a living being, as represented in its cylindrical form, in FIG. 2. The stent assembly 10 comprises a plurality of web-like, circumferential arrays 12, 12A, . . . 12H of switchback bends or loops or loops 14, generally in the manner of an arcuately shaped “hairpin-like” curve, as indicated within the dashed rectangle “X” shown in FIG. 1 and FIG. 3A.

There are, for example, a plurality of circumferential arrays of switchback loops or hairpin-like curves 12, 12A, 12B, 12C, 12D, 12E, 12F, 12G, and 12H spaced apart from one another along the longitudinal axis “L” of the stent assembly 10, as shown in FIGS. 1 and 2. The loops or bends 14 at a first end 16 of the stent assembly 10 in the first circumferential array 12 thereat are all generally in peripheral alignment with one another, as indicated by their edge in alignment with a border identified by a dashed line 11. In an embodiment, elongated loops 18 on the inwardly directed side of the first or leftmost circumferential array 12 of every third of the switchbacks or hairpin-like curves or loops 14 extend longitudinally beyond a peripheral border 15, while remaining loops 14 of the first or leftmost circumferential array 12 do not extend inwardly beyond the peripheral border 15. Further, the elongated loops 18 in each of the circumferential arrays 12, 12A etc. comprising, in an embodiment, at least every third of the switchbacks or hairpin-like curves or loops 14 can extend longitudinally beyond one or more of their peripheral border alignments, as indicated by dashed lines 15 and 21 of their adjacent bends, in an exemplary manner, for the two leftmost arrays 12 and 12A.

In an embodiment, a plurality of preferably smoothly curved, arcuate cross-links 50 are arranged so as to connect diagonally adjacent elongated loops 18 between longitudinally adjacent arrays 12, 12A, etc., of bends or curves 14. Those elongated loops 18 preferably comprise every third loop 14 as most easily seen in FIG. 1.

Loops (also referred to as curves) 14 are shown in an enlarged representation in FIG. 3A in an embodiment of the invention. The arcuately shaped “hairpin-like” curves 14 have a smoothly curved concave side 17 and a smoothly curved convex side 19. Thus, the concave and convex sides 17 and 19 of each curve 14 are configured to be curved circumferentially, that is, curved in the “same direction” or orientation, in their definition of each individual loop or curve 14.

In an embodiment, the second and successive circumferential arrays 12A, 12B etc, of switchback or hairpin-like curves or loops 14 are in generally corresponding longitudinal alignment with the switchback or hairpin-like curves or loops 14 of the first circumferential array 12 of loops 14 (shown in FIG. 1 as the leftmost array) at the first end 16 of the stent assembly 10, as indicated by dashed line CA, shown in FIG. 1 passing through the tips of the loops 14, which may be called “fronds” in conforming with a “Palm Tree” shape, described herein in greater detail. That is, a switchback or loop 14 of an Nth circumferential array 12N, for example, circumferential array 12D, is generally aligned according to a predetermined displacement, if any, with respect to loops 14 in the N+1 circumferential array 12N+1 of switchback or hairpin-like curves or loops 14, for example, circumferential array 12E. For example, beginning with array 12A, loops 14 are generally closely correspondingly aligned with loops 14 of alternating subsequent arrays 12B, 12D, etc. . . . , and offset (or out of phase) with respect to loops 14 of alternating subsequent arrays 12A, 12C, etc, thus providing a “Palm Tree” shape.

In an embodiment, each adjacent circumferential array 12, 12A, . . . 12H of loops or arcuately shaped hairpin-like curves 14 is joined to its longitudinally adjacent circumferential array 12A, 12B etc. . . . of loops or hairpin-like curves 14 by at least two smoothly curved arcuate cross-links 50. Each cross-link 50 extends from a mid-portion 52 of a curved section of an arch of an elongated switchback loop 18 to the tip portion 56 of the curved hairpin-like curve or bend 14 on a generally diagonally adjacent elongated curved switchback loop 18, as best represented in FIG. 1, and which is also illustrated in FIGS. 2, 5, and 6. Furthermore, each circumferential array is directly connected to each adjacent circumferential array by a cross-link 50 between a mid-portion 52 of an elongated curved switchback loop 18a of a first circumferential array, for example, array 12G and a tip portion 56 of an elongated curved switchback loop 18b, of an adjacent second circumferential array, for example, array 12F, that generally extends the arcuate curvature of the bend 18b leading to the tip portion 56. The direct connection by a cross-link 50 to a mid-portion 52 of a bend of a circumferential array promotes substantial coupling between any re-orienting, pivoting, and bending of a cross-link 50 with re-orienting, pivoting, and bending of that linked circumferential array, resulting in each circumferential array not generally being independently expandable with respect to an adjacent circumferential array and promoting even expansion across the stent assembly. Those cross-links 50 extending from tip portions 56 are on the same longitudinal end of a circumferential array 12, 12A etc and those cross-links that extend from a mid-portion 52 are on the opposite longitudinal end of the circumferential array, which can also help promote uniform expansion of the stent.

For various embodiments of the invention, the general pattern can be adapted for differently sized stents or stents of different strengths varied according to need. For example, the frequency or number of circumferential arrays may be varied and the number of hairpin-like curves or loops may be varied as necessary for each circumferential array. For example, embodiments of the pattern with six hairpin-like loops for each circumferential array can provide for a stent length of about 9 mm with four columns of circumferential arrays, a length of about 18 mm with 9 columns of circumferential arrays, a length of about 28 mm with 12 columns of circumferential arrays. These embodiments can have, for example, initial outer diameters of about 2 mm, crimped inner diameters of about 0.7 mm, and deployed outer diameters of about 2.75 mm, 3.0 mm, 3.5 mm, or 4.0 mm.

In other embodiments, the elongated switchback loops 18 in every series of peripherally adjacent bends of adjacent circumferential arrays extend longitudinally beyond the bends or tips of their circumferentially adjacent hairpin-like curves 14, as indicated by the dashed lines 15, 21, and 42, shown in FIG. 1.

In an embodiment, a generally semi-circumferentially extending annular, circumferentially elongated gap or space 30 between array 12 and longitudinally adjacent array 12A defined by their respective circumferential loops 14 and the arcuate cross-links 50 resembles the aforementioned branched “Palm Tree” configuration, most conspicuously shown in FIG. 1.

In an embodiment, the last circumferential array 12H of the stent assembly 10 has an edge array of bends 14 thereon which are generally in peripheral alignment with one another, as indicated by their common alignment with dashed line 40, as shown in FIG. 1. The last or rightmost circumferential array 12H at the second end 32 of the stent assembly 10 also has elongated bends or elongated switchback loops 18 that extend longitudinally beyond the peripheral edge of the adjacent switchback loops or hairpin-like curves 14 on that particular circumferential array 12H, as indicated by their extension in a longitudinal direction, “inwardly” beyond the dashed line 42, also shown in FIG. 1.

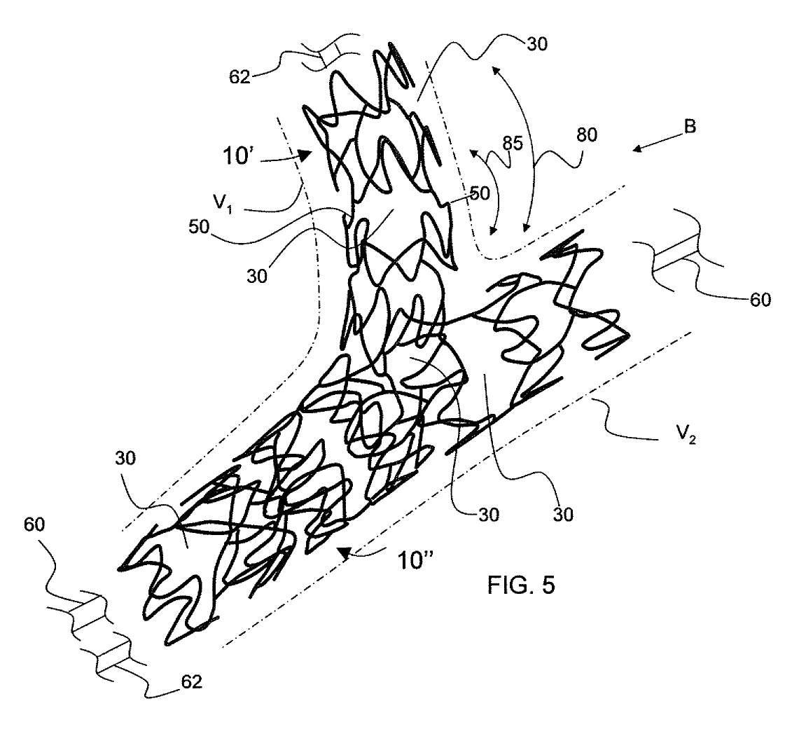

Thus, there are annular gaps 30 between adjacent circumferential arrays 12, 12A etc. of switchback loops or hairpin-like curves 14 comprising about 180 degrees (as represented by circumferential offset identifiers 64) of the peripheral space of the stent assembly 10 at that particular longitudinal location between adjacent arrays 12, 12A etc. The 180 degree clear, open, circumferentially disposed, “Palm Tree” shaped “open cell” space 30 between adjacent circumferential arrays 12, 12A etc. generally comprises a “half periphery” of the stent assembly 10, permitting a second stent assembly 10′ (see FIG. 5) to be passed therethrough and expanded outwardly as in a vessel bifurcation, because of the multiple longitudinally-dispersed, half-circumference “open cell” structure of each particular stent assembly 10, thereby allowing such multiple stent assembly interdigitation to be provided. Further embodiments within the scope of this invention can include more than two annular “open cell” spaces or gaps 30 between circumferential arrays 12, 12A etc of loops 14, depending upon the number of cross-links 50 dividing up each annular space between adjacent arrays 12, 12A etc. For example, one embodiment may extend the general pattern of open spaces 30 to comprise three annular “open spaces” or gaps 30, each one of which spans about a third of the periphery (about 120 degrees) of the stent assembly 10. In a further embodiment, a varying number (e.g. 2, 3 or more) of cross-links 50 may be disposed between adjacent arrays 12, 12A etc. is contemplated, to provide any particular desired variation in bending and/or in receptability to through-wall penetration by several stein assemblies 10, 10′ etc.

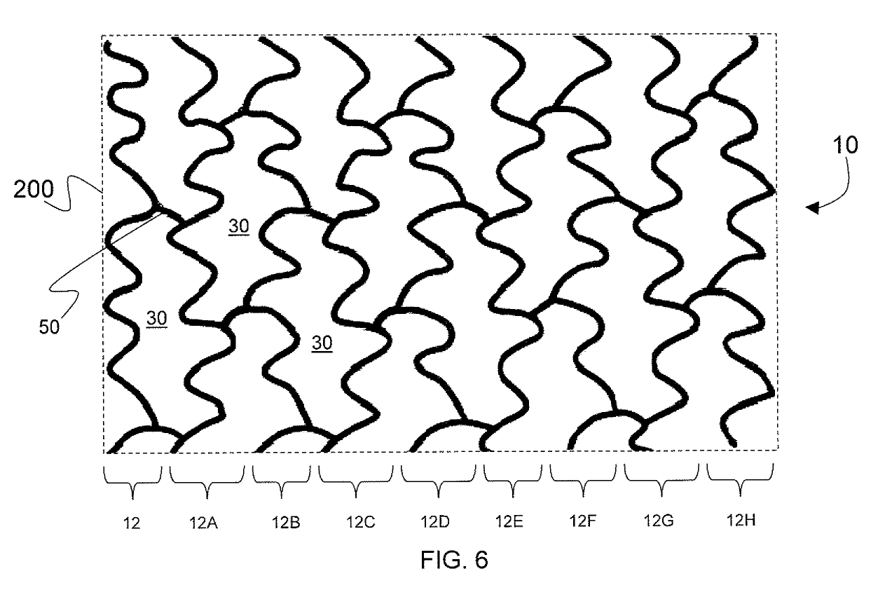

After the insertion of such a stent assembly 10 of the present invention in a vessel and upon expansion of the adjacent circumferential loops 14 of each array 12, 12A etc., each of the cross-links 50 between adjacent circumferential arrays 12, 12A, etc. may in an embodiment, be re-oriented slightly or pivoted, as viewed radially inwardly, indicated by the arrow “P” in FIG. 1. In an embodiment, as a stent assembly 10 expands from an unexpanded state such as shown in FIG. 1 to an expanded state (such as shown in FIG. 6), the cross-links 50 can rotate, pivot, and/or bend relative to the longitudinal axis “L” of the strut assembly so to be repositioned from an oblique orientation with respect to its alignment with the longitudinal axis “L” of the stent assembly 10 to an orientation which is more parallel to the longitudinal axis “L” of the stent assembly 10. Such a movement of these cross-links 50 assists in forestalling any shortening of the length of the stent assembly 10 as it expands within the vasculature of a patient. Such annular or circumferential disposition of the semi-circumferential gaps or spacings 30 during expansion of the stent assembly 10, and the rotation of the cross-links 50, however, remain in general circumferential disposed alignment with respect to the longitudinal axis of the stent assembly 10, and not obliquely angled with respect thereto. Such a stent assembly 10 foreshortening during expansion thereof can be, however, primarily prevented by the expansive common circumferential and longitudinally directed deformation of the curves or bends 14 due to their unique curvilinear configuration, which comprises the structure being moved radially outwardly.

The minimal number of cross-links 50 between longitudinally adjacent circumferential arrays 12, 12A etc of loops 14 adds to the stent assembly's flexibility and adaptability of that stent assembly 10 in the curved vasculature of a patient. Similarly, the untethered adjacent bends 14 in the respective circumferential arrays 12, 12A etc. allows for substantially uniform radial strength over the length of the stent assembly 10, permitting substantially uniform expansion and helps reduce or avoid such effects as “dog boning” or the foreshortening of the stent assembly 10 within a patient. In an embodiment, each of the cross-links 50 extending from a circumferential array 12A, 12B, . . . , 12E, is substantially circumferentially offset from each cross-link 50 extending from the same circumferential array on its longitudinally opposite side, thus providing flexibility and adaptability of that stent assembly 10 in the curved vasculature of a patient. In an embodiment, the circumferential offset is about 90 degrees as shown by circumferential offsets 54 between cross-links 50.

In an embodiment, the dimensions and geometry of the stent strut cross-sections, their relative orientation combined with the strut pattern, and the strut surface profile are designed to promote flexibility, to promote support of the vasculature, to minimize surface contact and damaging abrasion therefrom.

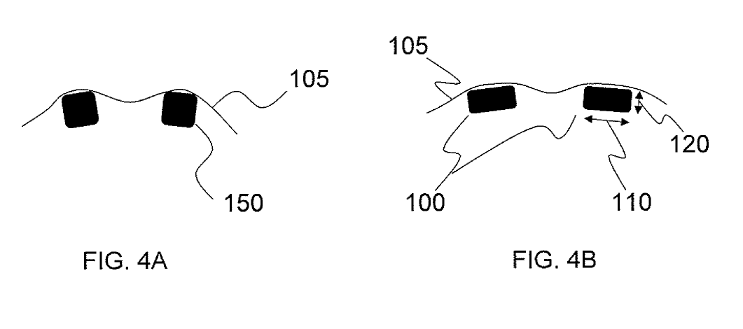

FIG. 3A is an enlarged illustrative view, in plan, of a portion of a circumferential array of arcuately shaped hairpin-like bends of the stent assembly shown in FIG. 1. FIG. 3B is an illustrative side perspective view of a strut section 100 of the assembly shown in FIG. 3A. FIG. 3C is an illustrative cross-sectional view across lines I-I′ of an embodiment of the stent strut section 100 shown in FIG. 3A. FIG. 4B is an illustrative cross-sectional view of a stent strut in accordance with an embodiment of the invention abutting a vessel wall 105. In an embodiment, a cross-sectional dimension 110 (defined herein as strut “surface width”) is generally planar relative to a targeted vessel surface and, in an embodiment, longer than its normal dimension 120 (defined herein as “strut height”). In contrast, FIG. 4A provides an illustrative cross-sectional view of a substantially square-shaped stent strut 150 abutting a vessel wall 105. The elongated dimension 110 as shown in FIGS. 3C and 4B provides a flatter, less angular, surface profile of the strut against a vessel wall than a more square profile such as of the strut 150 shown in FIG. 4A, thus reducing the potential for damage during stent expansion while retaining the necessary strength and flexibility to meet various biomechanical requirements of an expandable stent. In an embodiment of the invention, dimension 110 of the stent strut 100 is between about 90 to about 130 microns and dimension 120 of the stent strut 100 is between about 50 to about 80 microns and suitable, for example, for smaller vessels (i.e., less than 3 mm in diameter). In an embodiment, dimension 110 averages about 115 microns across stent 10 and dimension 120 averages about 65 microns across the struts of stent 10. In an embodiment of the invention, dimension 120 of the struts 100 is of a thickness of between about 60 and 100 microns which can be suitable, for example, for medium sized vessels (i.e., from 3 mm to less than 4 mm in diameter). In an embodiment, dimension 110 is at least about one and a half times that of dimension 120 and, in an embodiment, about twice or more than that of dimension 120.

Referring now to FIG. 5, the interdigitation of a second stent assembly 10′ (extending along axis 300) through a first stent assembly 10″ (extending along axis 350) within a bifurcated body vessel B is shown in an embodiment of the invention. Such a multiple stenting is made easier by virtue of the expansive circumferential “Palm Tree” shaped open cell spaces 30, such as described herein with regard to the embodiments illustrated in FIG. 1. A minimal number of cross-links 50 (e.g., two cross-links) between adjacent arrays 12, 12A, etc of hairpin-like curves 14 promotes the curvature of each stent 10′ and 10″ for accommodating one another, and wherein a first stent 10′ can be penetrated by another stent 10′ without significant interference, which is highly beneficial to a patient needing such a bifurcation procedure. This double stenting at a bifurcated vessel B can be achieved one stent at a time, with the second stent assembly 10′ being directed though the longitudinal opening of the first stent assembly 10″ then angularly directed through such a “Palm Tree” shaped side opening 30 which is in alignment with vessel V₁ of the bifurcated vessel B being stented. Further, the second stent assembly 10′ in a bifurcation procedure of the present invention may be of shorter length or of smaller diameter to facilitate the stenting of a bifurcation B, or to accommodate only a relatively short or narrow branch requiring stenting extending from the parent vessel V₂, to minimize any unnecessary overlap between the first and second stent assemblies 10″ and 10′.

In addition, the limited number of cross-links 50, their distribution, and the “flat” strut profile (such as that illustrated in FIGS. 3C and 4B) provides adequate vessel support and also provides longitudinal flexibility in a highly curved area such as bifurcating vessel V₁. The “flat” strut profile shown in FIG. 3C reduces the tendency of struts, including cross-links 50, to bend along the 110 dimension and rather promotes bending along the narrower 120 dimension, and helps limit a further longitudinal widening of already “open” areas 30 along areas of high vascular curvature. In an embodiment, each cross-link 50 has a “flat” profile and is substantially circumferentially offset from every other cross-link 50 extending from the opposite side of the circumferential array. In an embodiment, the offset is about 90 degrees, as shown in the assembly of FIGS. 1 and 2.

Referring again to FIG. 5, where stent 10′ is shown partially inserted into a bifurcated vessel V₁, the curved arrow 80 is shown generally representing the overall curvature of bifurcating vessel V₁. The resulting curvature of stent 10′ in response to overall curvature 80 of bifurcating vessel V₁ is concentrated more so along the section generally defined by curved arrow 85, where a cross-link 50 is generally circumferentially oriented with the overall curvature 80. The “flat” strut profile and alternating nature of the circumferential position of the cross-links 50 helps promote bending in this manner, thereby reducing excessive widening of open areas 30. Thus, in an embodiment, the bending of a stent in response to the curvature of a vessel tends not to excessively longitudinally widen an open area 30, further helping prevent a collapse of tissue and preventing bending of the stent by operation of other factors (e.g., calcification) not generally associated with the overall natural shape of a vessel.

In embodiments of the invention, various multiple-stent deployments such as in accordance with assembly FIG. 5 or, for example, a “kissing stent” procedure (in which a first stent is “extended” by placing a second stent at least partway through a longitudinal end of the first stent) are more fully described in co-pending and related U.S. patent application Ser. No. 11/613,443, filed on Dec. 20, 2006 and entitled “Flexible Expandable Stent,” the entire contents of which is incorporated herein by reference in its entirety. The flexible, broadly supportive, and “open” characteristics of a stent assembly 10, for example, in accordance with an embodiment of the invention is well adapted for placement with one or more other stent assemblies, such as one or more stents in a localized vessel area.

While providing substantially evenly distributed support of a vessel wall, an embodiment of the invention provides a strut-to-vessel contact percentage of less than about 14%.

FIG. 6 is a view of an enlarged flattened illustrative pattern of the stent 10 of FIGS. 1 and 2 shown after balloon expansion. In an embodiment, after balloon expansion, the stent 10 is expanded to about twice its original diameter. The expanded pattern illustrates the limited longitudinal widening of open areas 30 which occurs after a stent expansion and deformation of the entire cell 30 and co-dependent expansion between circumferential arrays 12, 12A, 12B, etc. described further herein. A strut-to-vessel contact ratio is the percentage of strut-to-vessel contact across an area of the vessel surface encompassing the stent and, for example, can be represented in FIG. 6 as the percentage of the surface area occupied by the struts of stent 10 over the total area represented by box 200. In an embodiment, a strut-to-vessel contact percentage ranges from about 10.5 percent or less to about 13.5 percent or less, being generally proportional to expansion diameters ranging between about 2.75 and 4 millimeters. In an embodiment, a stent in accordance with the pattern of FIG. 1 has a strut dimension (or surface width) 110 (described above, for example, in connection with FIGS. 3B, 3C, and 4B) averaging about 115 microns and, at an expanded diameter of about 3 millimeters, would have a strut-to-vessel contact percentage of about 11%.

Various embodiments in accordance with the invention can provide well distributed vessel support combined with well distributed deformation upon expansion, thus helping avoid concentrated abrasion and excessive damage to particular vessel areas. Comparing FIGS. 1 (an unexpanded stent) and FIG. 6 (an expanded stent), for example, the struts of stent 10, including cross-links 50, will collectively bend, pivot, and deform together to the general orientation as shown in FIG. 6. In an embodiment, as stent 10 is expanded, the longitudinal ends of circumferential arrays 12, 12A, etc., generally remain proximal to each adjacent circumferential array 12, 12A, etc., while also substantially avoiding foreshortening. By distributing bending and pivoting over a substantial portion such as over the stent 10 during expansion, excessive movement and abrasion is significantly avoided.

Furthermore, when a cross-link 50 is pivoted in a more longitudinal orientation, its direct connection to the mid-portion 52 of a curved section of arch tends to occur in conjunction with a bending of a switchback loop 18 rather than a solely longitudinal widening of an open area 30. Limiting the further longitudinal opening of these areas generally maintains relatively consistent vessel support around areas 30 and helps avoid excessively large unsupported areas that can be problematic with typical “open” stent designs.

Referring again to FIG. 3A, the direction of loops or curves 14 substantially reverse through bends 60 and 62 in a switchback hairpin-like manner and illustrates exemplary areas 20 and 27 of stent 10 that have, in an unexpanded state, relatively greater (or tighter) degrees of curvature than other areas of the stent. In an embodiment of the invention, a minimal radius of curvature along the entire surface of the unexpanded stent (that is, not expanded beyond a point generally appropriate for deployment), including along those areas of highest curvature, is about 65 microns. In an embodiment of the invention, the minimal radius of curvature is about 80 microns. In an embodiment, the stent has one or more layers of coating material while having a minimal radius of curvature of about 50 microns.

The relatively large minimum radius of curvature of the unexpanded stent provides a highly favorable surface over which coating materials can be deposited. Distributing curvature more evenly over the entire stent helps avoid the inclusion of areas of excessively tight curvature that promote the disadvantages of coating prior designs. For example, the overall openness of the curves 14 helps avoid a structural blockage that could prevent a consistent coating over the entire stent surface. A tightly closed area of curvature may more likely receive less material than other areas not similarly closed, thus resulting in insufficient coating about the tightly closed areas.

An inconsistent coating process may prompt thicker layers of material to be applied overall to the stent surface in order to ensure adequate coverage overall. Thicker layers of material, particularly metallic material, can detrimentally effect biomechanical properties of the stent, including flexibility and tissue-to-stent surface contact. In addition, the areas of relatively low curvature help avoid the effect of “webbing,” wherein an area of tight curvature acting as a crevice can essentially be filled in and could cause the coating to stretch apart and/or split during expansion of the stent, including the area of tight curvature. Moreover, areas of tight curvature (with our without coatings) can be subject to greater mechanical stresses when they are opened (such as during expansion), thus increasing the likelihood of metal fatigue, fractures, and/or increased post-expansion recoil.

In a further embodiment of the invention, opposing surfaces of a stent (e.g., in accordance with the design of FIGS. 1 and 2) are separated by a minimal distance in order to enhance surface modification processes and help avoid issues such as, for example, uneven coating, webbing, and/or cracking. Referring to FIG. 3A, exemplary straight-line normal spans 70 are shown between opposing strut surfaces and, in an embodiment, are of at least this minimal distance. In an embodiment, all opposing surfaces of the stent structure are separated by normal straight-line distances (or spans) by a minimal distance of about 130 microns. In another embodiment, all normal straight-line distances (or spans) of opposing surfaces (e.g., normal spans 70) have a minimal distance of about 160 microns.

The “open” curvature and/or substantially non-interfering characteristics of various embodiments of the invention promote a structure conducive to various coating technologies including, in particular, those involving streams of coating particles and/or bombarding particles directed at the surface of an embodiment (e.g. the struts of annular arrays 12, 12A etc. and cross-links 50) of the stent structure. In an embodiment of the invention, the coating process comprises directing a stream of particles (e.g. coating and/or bombarding atoms or ions) toward the stent structure. In an embodiment of the invention, a coating process comprises at least one of electrochemical deposition, chemical-vapor deposition, electroplating, electro-polishing, ion-assisted deposition, and/or ultrasonic spraying.

In an embodiment, the struts are layered with inert biocompatible materials, including gold, silver, platinum, and/or various non-metallic materials.

In an embodiment of the invention, one or more layers is provided with an ion-assisted deposition onto the stent structure, such as, for example, through methods which use one or more magnetrons such as described in pending U.S. patent application Ser. No. 09/999,349, filed Nov. 15, 2001, entitled published Sept. 26, 2006 as US Patent Application Publication Number 2002/-0138130A1 and pending U.S. patent application Ser. No. 11/843,376, filed Aug. 22, 2007, and U.S. patent application Ser. No. 11/843,402, filed Aug. 22, 2007, published Sep. 4, 2008, the contents of each of which are incorporated herein by reference in their entirety. Various embodiments of these devices and methods involve actively and/or passively biasing a substrate with electrical charge and thus increasing the attraction of charged coating and/or bombarding atoms or ions, for which various embodiments of the present invention can help improve the uniformity of the magnetic attraction.

In an embodiment, the struts of annular arrays 12, 12A, etc. and cross-links 50 are comprised of a highly radiopaque substrate such as, for example, cobalt-chromium material, stainless-steel, and nitinol material. In a further embodiment, such as in accordance with previously cited and incorporated U.S. patent application Ser. No. 11/843,376, gradations of platinum and palladium ions are implanted onto a cobalt chromium base through various embodiments of these methods to produce an adhesion layer of essentially palladium or gold, a transition layer of increasing platinum content and decreasing palladium content and a bio-compatible metal capping layer of essentially platinum or having, at least, a predominance of platinum. In further embodiments of the present invention, the palladium and platinum layers can be from about 100 angstroms and up to about 5,000 angstroms thick, preferably greater than for example, about 500 angstroms thick, and less than about 2,500 angstroms, such that they are optimized to maximize the smoothness and stability of the layers. The thicknesses may depend upon various parameters, including the size and projected expansion of the stent assembly.

In an embodiment, the metal capping layer is manufactured with at least one of platinum, platinum-iridium, tantalum, titanium, tin, indium, palladium, gold and alloys thereof. In an embodiment, the metal capping layer and all layers within the metal capping layer (such as, for example, an adhesion layer, or no layers between the substrate and metal capping layer) have a combined thickness of less than about a micron. In an embodiment, the metal capping layer and all layers within the metal capping layer have a combined thickness of less than about 0.5 microns. In an embodiment, the metal capping layer and all layers within the metal capping layer have a combined thickness of less than about 0.25 microns.

In an embodiment, surface modifications are applied to struts of annular arrays 12, 12A etc. and cross-links 50 that provide textured surfaces such as in accordance with previously cited and incorporated U.S. patent application Ser. No. 11/843,402. The texturing can improve the surface of the stent for purposes of encouraging healthy endothelial growth upon deployment, providing a more adhesive surface for additional layers such as polymers having drug-eluting properties, and/or improving the retention and avoiding undesired slippage between the surface of the stent and a delivery system (e.g. a balloon catheter) during delivery.

It will be understood by those with knowledge in related fields that uses of alternate or varied materials and modifications to the methods disclosed are apparent. This disclosure is intended to cover these and other variations, uses, or other departures from the specific embodiments as come within the art to which the invention pertains.

1. A flexible, expandable, elongated stent assembly comprising a pattern of interconnected struts along a curvilinear path, the struts defining a generally cylindrically shaped channel that extends along a longitudinal axis, the channel having a plurality of openings, the struts comprising a plurality of circumferential arrays of webs or bends of a material, each circumferential array connected to an adjacent circumferential array by fewer than four cross-links, wherein each cross-link extending from a first side of a circumferential array of the plurality of circumferential arrays is substantially circumferentially offset from every cross-link extending from an opposite side of the same circumferential array, and wherein each of the struts has, in a cross-section generally normal to the curvilinear path of the strut and normal to a center of curvature of the channel, a strut surface width that is at least one and a half times that of a strut surface height of the strut.

2. The flexible, expandable stent assembly of claim 1, wherein each circumferential array is connected to an adjacent array by two and only two cross-links.

3. The flexible, expandable stent assembly of claim 1, wherein the cross-links are arranged such that, upon expansion of said stent assembly from a first position in an unexpanded state to a second position in an expanded state, the cross-links are re-oriented or pivoted with respect to said longitudinal axis.

4. The flexible, expandable, stent assembly of claim 1, wherein each of the cross-links is attached to a bend of a circumferential array such that any bending or pivoting of the each of the cross-links is directly and substantially coupled with a bending or pivoting of said attached circumferential array.

5. The flexible, expandable stent assembly of claim 4, wherein each of said cross-links extends from a mid-portion of a longitudinally extending curved section of a first bend of a first array to a tip portion of a second bend of a longitudinally adjacent second array.

6. The flexible, expandable stent assembly of claim 4, wherein the first bend of the first array is positioned diagonally from the second bend of the second array.

7. The flexible, expandable stent assembly of claim 1, wherein each cross-link extending from the first side of each circumferential array is substantially circumferentially offset by at least about 60 degrees from said every cross-link extending from an opposite side of the same circumferential array.

8. The flexible, expandable stent assembly of claim 7, wherein each cross-link extending from the first side of each circumferential array is offset by about 90 degrees from the cross-links extending from the opposite side of the circumferential array.

9. (canceled)

10. (canceled)

11. The flexible, elongated stent assembly of claim 1, wherein said surface width is between about 90 and 130 microns.

12. The flexible, elongated stent assembly of claim 11, wherein said surface width is about 120 microns.

13. The flexible, elongated stent assembly of claim 1, wherein said strut surface width is about twice that of the strut surface height.

14. The flexible, elongated stent assembly of claim 1, wherein the stent assembly is manufactured such that, upon having an expanded diameter of between about 2.75 mm and 4 mm in a vessel, the stent assembly has less than about 10.5% to 13.5% strut-to-vessel contact over an area encompassing an entire periphery of the channel.

15. The flexible, elongated stent assembly of claim 1, wherein the circumferential arrays are comprised of a collection of circumferential arrays of switchback webs or bends, wherein the circumferential arrays are connected to one another by an arrangement of cross-links, wherein, from a flattened radially-directed view, each of the webs or bends is defined by a path of an arc, and each of the cross-links defined by a path of of an arc that follows and continuously extends the same path of an arc that defines at least one of said webs or bends.

16. The flexible, expandable stent assembly of claim 15, wherein a substantial portion of each of said arcuately shaped, generally hairpin-like curved webs or bends form arcs of generally the same concavity with respect to the circumference of said stent assembly.

17. (canceled)

18. (canceled)

19. The flexible, expandable stent assembly of claim 1, wherein a first stent assembly of the flexible, expandable stent assembly is combined with a second stent assembly that extends at least partway through an opening of the first stent assembly, and wherein the second stent assembly extends at least partway through a generally circumferentially disposed opening of the first stent assembly.

20. (canceled)

21. (canceled)

22. The flexible, expandable stent assembly of claim 1, wherein each circumferential array is not independently expandable in the radial direction.

23. The flexible, expandable, stent assembly of claim 22, wherein each one of the cross-links is fixed to a bend of a circumferentially array such that any bending or pivoting of one of the cross-links is directly and substantially coupled with a bending or pivoting of said bend of a circumferential array.

24. (canceled)

25. A method for expanding and supporting the vasculature of a patient, the method comprising the steps of:
placing a first stent assembly into a vessel of the patient, the stent assembly comprising a pattern of interconnected struts along a curvilinear path, the struts defining a generally cylindrically shaped channel that extends along a longitudinal axis, the channel having a plurality of openings, the struts comprising a plurality of circumferential arrays of webs or bends of a material, wherein each cross-link extending from a first side of a circumferential array of the plurality of circumferential arrays is substantially circumferentially offset from every cross-link extending from an opposite side of the same circumferential array, each circumferential array connected to an adjacent circumferential array by fewer than four cross-links, and wherein each strut has, in a cross-section generally normal to the curvilinear path of the strut and normal to a center of curvature of the channel, a strut surface width that is at least one and a half times that of a strut surface height of the strut.

26. (canceled)

27. (canceled)

28. (canceled)

29. (canceled)

30. The method of claim 25, wherein each one of the cross-links is fixed to a bend of a circumferentially array such that the re-orienting or pivoting of the each one of the cross-links is directly and substantially coupled with a bending or pivoting of said bend of a circumferential array.

31. (canceled)

32. The method of claim 25, wherein the stent is expanded to a diameter between about 2.75 millimeters and 4 millimeters and provides an overall strut-to-vessel contact percentage of less than about 10.5% to 13.5% of vessel area encompassing the periphery of the channel.

33. The method of claim 25, wherein the stent conforms to curves in the vessel of the patient by generally concentrating a bending of the stent about the longitudinal axis over portions of the stent along the longitudinal axis where the circumferential position of the cross-links substantially corresponds to apexes of said curves in the vessel.

34. The method of claim 25, wherein each cross-link extending from the one side of each circumferential array is offset by about 90 degrees from said every cross-link extending from the opposite side of the circumferential array.

35. (canceled)

36. The method of claim 35, wherein the strut surface width is between about 90 and 130 microns.

37. (canceled)

38. The method of claim 25, wherein said the strut surface width is of about twice that of the strut surface height.

39. The method of claim 25, wherein the structure of struts of the stent assembly comprises a plurality of circumferential arrays of switchback webs or bends, wherein the circumferential arrays are connected to one another by an arrangement of cross-links, wherein, from a flattened radially-directed view, each of the webs or bends is defined by a path of an arc, and each of the cross-links is defined by a path of of an arc that follows and continuously extends the same path of an arc that defines at least one of said webs or bends.

40. (canceled)

41. (canceled)

42. (canceled)

43. The method of claim 42, wherein the first stent assembly is placed across a first arm of a bifurcated vessel and wherein the second stent assembly is placed at least partway through a side wall opening of said first stent assembly and into a second arm of the vessel bifurcation.

44. (canceled)

45. (canceled)

46. (canceled)

47. (canceled)
